(19) United States
(12) Patent Application Publication
(10) Pub. No.: US 2018/0344960 A1
(43) Pub. Date: Dec. 6, 2018

(54) SECURE ENDOTRACHEAL TUBE HOLDER

(71) Applicant: Maya Kaneyasu, Seattle, WA (US)

(72) Inventor: Maya Kaneyasu, Seattle, WA (US)

(21) Appl. No.: 15/997,166

(22) Filed: Jun. 4, 2018

Related U.S. Application Data
(60) Provisional application No. 62/515,955, filed on Jun. 6, 2017.

(51) Int. Cl. A61M 16/04

(52) U.S. Cl. CPC A61M 16/049; A61M 2205/584; A61M 2205/583; A61M 2240/00

(57) ABSTRACT

A secure endotracheal tube holder is provided that is specifically designed to securely hold an endotracheal tube in the neonatal population and that provides stability when used with tape. It is easy to use and replaces non-approved devices such as the post manufacture-modified umbilical cord clamp, which were not intended for securing the endotracheal tube or other products on the market that have high extubation rates. The taberculated inside surfaces of the opening hold the tube in place while a lateral curve provides for easier handling when securing to the face with tape or when the tube requires adjustment. The device is clearly marked with appropriate numeric size indicating the tube size along with a color scheme for visual recognition, and it is constructed from clear or colored plastic to facilitate seeing the markings and depth.

### BACKGROUND

### Technical Field

The present disclosure is related to endotracheal tubes and, more particularly, to a device for holding an endotracheal tube in a neonatal application and method of using the same.

### Description of the Related Art

Neonates that are born preterm are at high risk for respiratory failure secondary to immature lungs and decreased thoracic musculature to have sustained and effective breathing. Often, seconds or minutes after delivery, preterm neonates require a provider who is skilled in neonatal resuscitation so that an endotracheal tube (ETT) can be placed correctly in the trachea. This is often performed by a neonatologist or neonatal advance registered nurse practitioner who will insert a laryngoscope into the oropharynx (mouth) and look down directly into the opening of the trachea. Once the landmark such as the epiglottis is visualized, the ETT is passed through the vocal cords. The end of the ETT is then connected to an oxygen source so that pressure and oxygen can be effectively administered directly into the trachea and into the lungs. This modified oxygen delivery system assists preterm neonates in respiratory failure or distress in more effective gas exchange.

There are many ETT holders in the market for adults, but few are designed to securely hold ETTs in the neonatal population. Of the products that are manufactured for neonates, historically, many have high rates of accidental extubations (i.e., inadvertent dislodgment of the ETT out of the trachea) that can cause respiratory compromise in this fragile population. Hence, many staff (registered nurses, respiratory therapist, and doctors) have resorted to using various forms of tape to secure ETT in neonates. This is often seen in conjunction with unauthorized modifications to an umbilical cord clamp in order to provide more stability.

### BRIEF SUMMARY

The present disclosure is directed to a device to aid in holding an endotracheal tube in a neonatal application. In accordance with one aspect of the present disclosure, a device is provided that includes a first arm having a curvilinear planform shape, a first end, an opposing second end having a first connecting component, and a first clamp component located between the first end and the second end. The device further includes a second arm having a curvilinear shape that is sized and shaped to enable the second arm to be parallel to the first arm in a nested relationship, a first end, and an opposing second end having a second connecting component that is capable of cooperating with the first connecting component to releasably connect the second end of the second arm to the second end of the first arm. The second arm further includes a second clamp component located between the first end and the second end that is capable of aligning with the first clamp component to form an opening sized and shaped to clamp the endotracheal tube in place in the opening. The second arm is rotatably coupled to the first arm to enable movement of the first and second arms between an open position, in which the second arm is not in a nested relationship with the first arm, and a closed position, in which the first and second arms are in the nested relationship and in which the first connecting component is connected to the second connecting component to hold the device in the closed position.

In accordance with another aspect of the present disclosure, at least one of the first and second clamp components, and preferably both of the clamp components has a gripping structure on an inside surface, ideally in the form of a taberculated structure or surface, to grip the endotracheal tube when the device is in the closed position.

In accordance with a further aspect of the present disclosure, the first arm is pivotally associated with the second arm at a connection point adjacent to the first ends of the first and second arms to enable the second end of the first arm to move into and out of connecting engagement with the second end of the second arm.

In accordance with still yet another aspect of the present disclosure, the first connecting component is a post and the second connecting component is a C-shaped opening that is sized and shaped to snap over the post and retain the device in the closed position.

In accordance with another aspect of the present disclosure, the first and second arms each have a substantially linear center section that connects the respective first ends to the second ends, and the device further comprises an axel extending through the center section adjacent the first end of each of the first and second arms that provides a pivotal association between the first and second arms.

In accordance with an additional aspect of the present disclosure, the first clamp component comprises a first portion of the opening that opens towards a first direction and the second clamp component comprises a second portion of the opening that opens towards a second direction that is opposite the first direction when the first and second arms are in the closed position.

In accordance with a further aspect of the present disclosure, the first and second ends of each of the first and second arms are enlarged with respect to the respective center sections of the first and second arms to provide anchor points.

In accordance with another implementation of the present disclosure, a system is provided that includes an endotracheal tube, a device for holding the endotracheal tube as described above, and a securing or fastening means for attaching the device for holding the endotracheal tube to a patient with the endotracheal tube placed into the trachea of the patient.

As will be readily appreciated from the foregoing, a secure endotracheal tube holder is provided that is specifically designed to hold an endotracheal tube securely in the neonatal population and that provides stability when used with tape. It is easy to use and would replace the use of non-approved devices such as the post manufacture-modified umbilical cord clamp, which were not intended for securing the endotracheal tube or other products on the market that have high extubation rates. Moreover, the taberculated inside surfaces of the opening hold the tube in place while a lateral curve provides for easier handling when securing to the face with tape or when the tube requires adjustment. The device is clearly marked with appropriate numeric size indicating the tube size along with a color scheme for visual recognition, and it is constructed from clear or colored plastic to facilitate seeing the markings and depth.

### BRIEF DESCRIPTION OF THE SEVERAL VIEWS OF THE DRAWINGS

The foregoing and other features and advantages of the present disclosure will be more readily appreciated as the same become better understood from the following detailed description when taken in conjunction with the accompanying drawings, wherein:

FIG. 1 is an illustration of one known umbilical cord clamp used for holding an endotracheal tube;

FIG. 2 is an illustration of the umbilical cord clamp of FIG. 1 holding an endotracheal tube adjacent the mouth of a patient;

FIG. 3 is an illustration of one method of fastening the umbilical cord clamp with endotracheal tube to the face of the patient using tape;

FIG. 4 is a front elevational view of a device for holding an endotracheal tube formed in accordance with the present disclosure and in which the device is in a closed configuration;

FIG. 5 is a front elevational view of the device of FIG. 4 in an opened configuration; and

FIG. 6 is a top plan view of the device of FIG. 4 in the closed configuration with an endotracheal tube held in place on the device.

### DETAILED DESCRIPTION

In the following description, certain specific details are set forth in order to provide a thorough understanding of various disclosed implementations. However, one skilled in the relevant art will recognize that implementations may be practiced without one or more of these specific details, or with other methods, components, materials, etc. In other instances, well-known structures or components or both associated with the use of endotracheal tubes, including tape, pumps for air or fluid, connection mechanisms, and the structure of internal voids with and without air or fluid channels have not been shown or described in order to avoid unnecessarily obscuring descriptions of the implementations.

Unless the context requires otherwise, throughout the specification and claims that follow, the word “comprise” and variations thereof, such as “comprises” and “comprising” are to be construed in an open inclusive sense, that is, as “including, but not limited to.” The foregoing applies equally to the words “including” and “having.”

Reference throughout this description to “one implementation” or “an implementation” means that a particular feature, structure, or characteristic described in connection with the implementation is included in at least one implementation. Thus, the appearance of the phrases “in one implementation” or “in an implementation” in various places throughout the specification are not necessarily all referring to the same implementation. Furthermore, the particular features, structures, or characteristics may be combined in any suitable manner in one or more implementations.

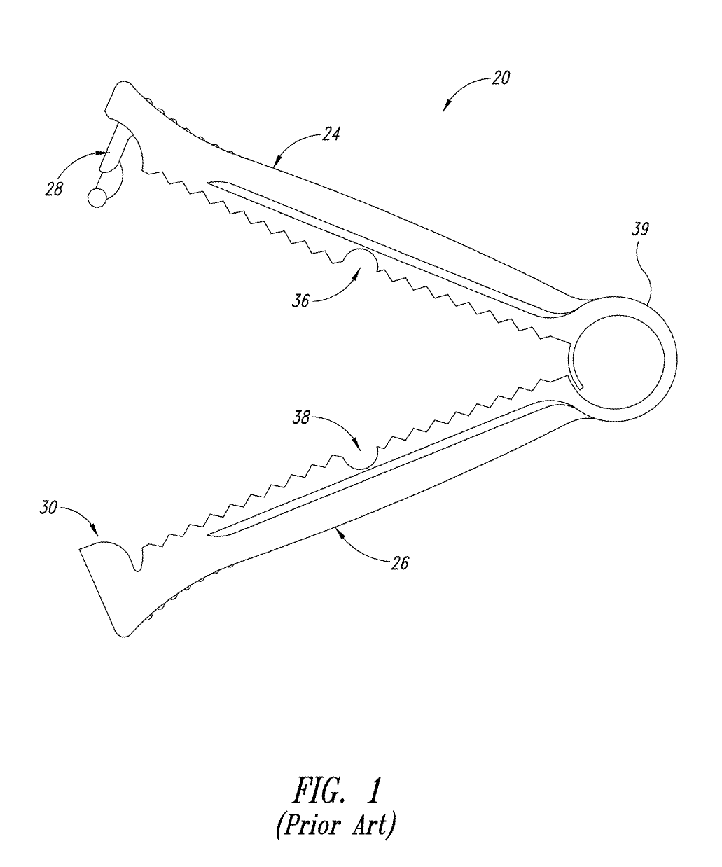

Referring initially to FIGS. 1 to 3, a conventional umbilical cord clamp 20 is shown as modified for use with an endotracheal tube 22. The typical umbilical cord clamp 20 is a unitary piece of resilient material with first and second jaw members 24, 26 that clamp together at one end with a clamping mechanism 28, 30. The jaw members 24, 26 have an elongate linear shape with teeth 32 formed on the interior surfaces of the first and second jaw members 24, 26 to clamp the umbilical cord therebetween.

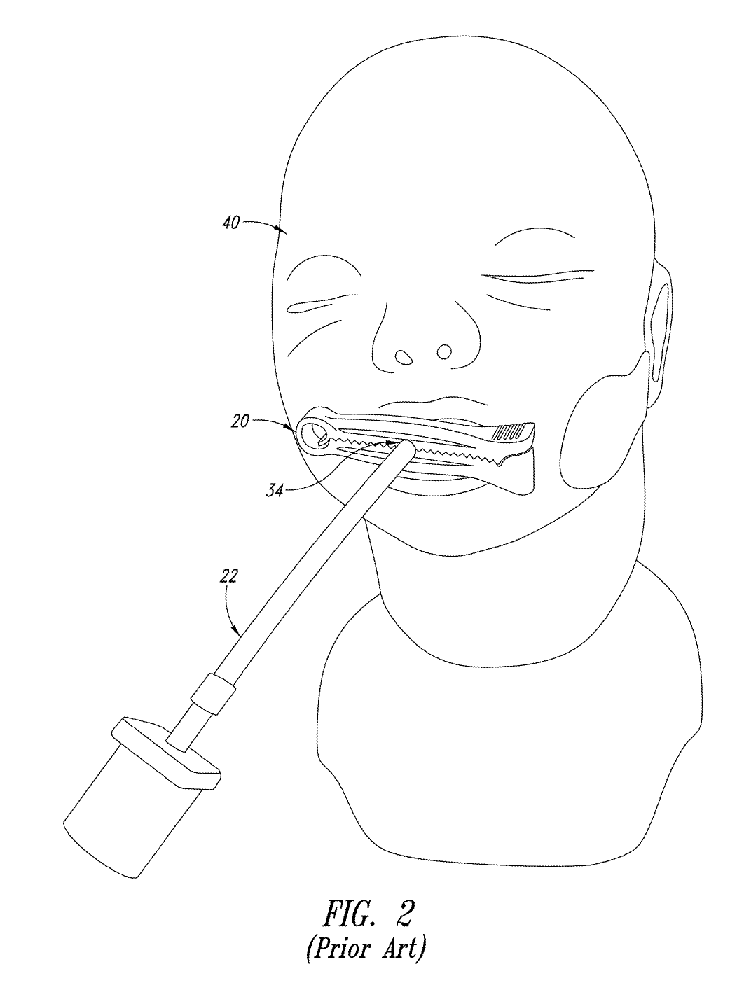

Medical staff and doctors modify this device by drilling a hole 34 in the first and second jaw members 24, 26 that is formed by a first semicircular opening 36 and second semicircular opening 38. The tube 22 is clamped into the opening 38 by the first and second jaw members 24, 26, as shown in FIG. 2 for placement of the tube 22 in the trachea of a neonatal patient 40. FIG. 3 illustrates the use of tape 42 to fasten the umbilical cord clamp 20 to the patient's face. One drawback presented by the use of an umbilical clamp 20 is the linear shape of the jaw members 24, 26, making it uncomfortable and unstable when fastened to the patient's face. Additional force is required from the tape 42 to keep the umbilical cord clamp 20 stationary. Another drawback is the tube 22 tends to slide inside the opening 38. A further drawback is the use of compliant material with a fixed hinge point 39 that biases the arms 24, 26 together or apart, depending on the design, requiring the use of force to either open or close the arms and clamp the tube 22 in place.

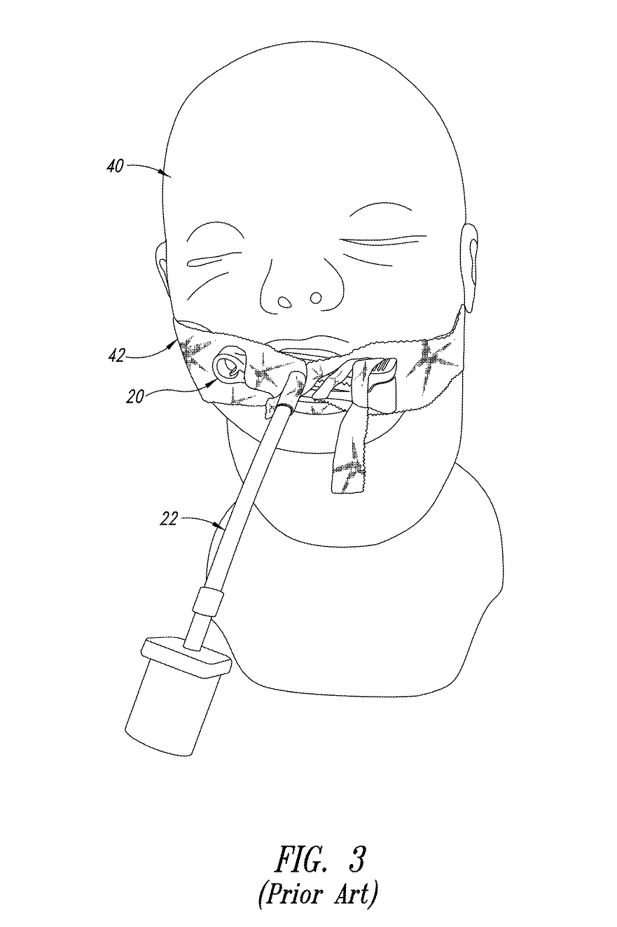

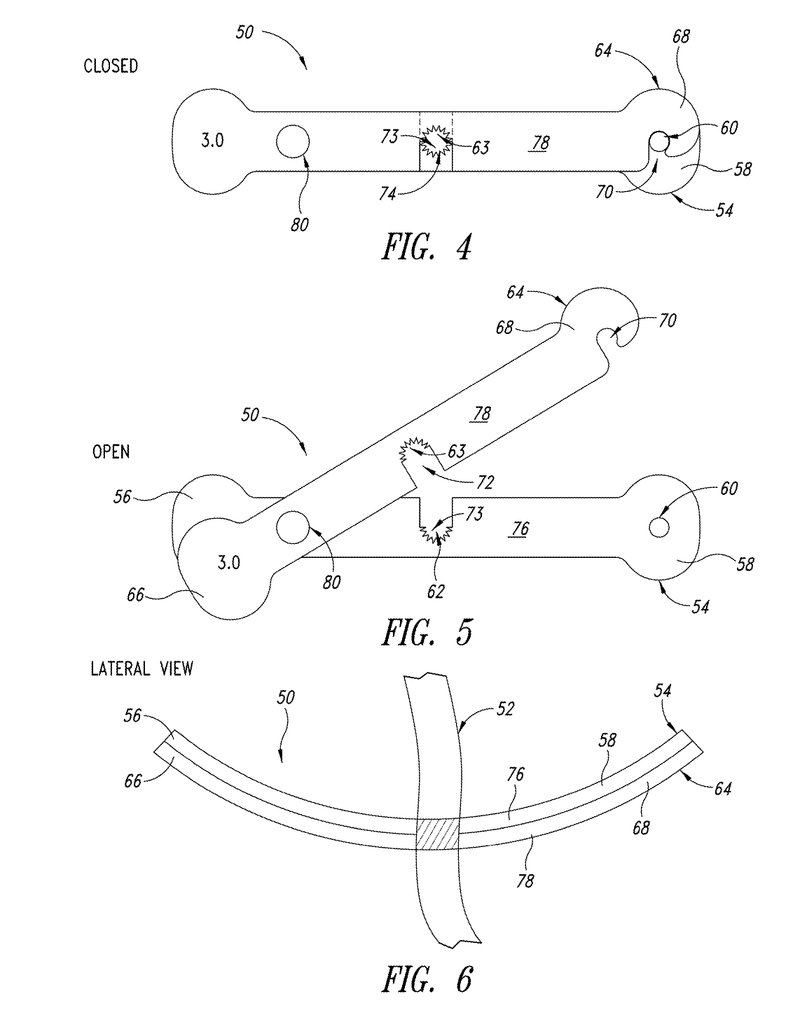

Referring next to FIGS. 4-6, shown therein is one implementation of a device 50 to aid in holding an endotracheal tube 52 in a neonatal application. Neonatal refers to babies up to 4 weeks old. The device 50 can be used in any infant in an ICU setting, whether it be in the Neonatal or Pediatric Intensive Care Unit, which requires a breathing tube. In accordance with this representative design, the device 50 includes a first arm 54 having a curvilinear planform shape, a first end 56, an opposing second end 58 having a first connecting component 60, and a first clamp component 62 located between the first end 56 and the second end 58. The device 50 further includes a second arm 64 having a curvilinear shape that is sized and shaped to enable the second arm 64 to be parallel to the first arm 54 in a nested relationship, a first end 66, and an opposing second end 68 having a second connecting component 70 that is capable of cooperating with the first connecting component 60 to releasably connect the second end 68 of the second arm to the second end 58 of the first arm 54. The second arm 64 further includes a second clamp component 70 located between the first end 66 and the second end 68 that is capable of aligning with the first clamp component 60 to form an opening 74 sized and shaped to clamp the endotracheal tube 52 in place in the opening 74.

The second arm 64 is rotatably coupled to the first arm 54 to enable relative movement of the first and second arms 54, 64 between an open position (shown in FIG. 4), in which the second arm is not in a nested relationship with the first arm, and a closed position (shown in FIGS. 4 and 6), in which the first and second arms 54, 64 are in the nested relationship and in which the first connecting component 60 is connected to the second connecting component 70 to hold the device 50 in the closed position.

The first arm 54 is pivotally associated with the second arm 64 at a connection point adjacent to the first ends 56, 66 of the first and second arms 54, 64 to enable the second end 58 of the first arm 54 to move into and out of connecting engagement with the second end 68 of the second arm 64 or vice versa. In a preferred implementation, the first and second arms 54, 64 each have a substantially linear respective center section 76, 78 that connects the respective first ends 56, 66 to the second ends 58, 68. The lateral curve formed into the first and second arms 54, 64 provides for easier handling when securing the device 50 to the face with tape or when the tube 52 requires adjustment. This decreases the amount of force needed to be applied by the tape to the patient's face, increase comfort while increasing stability of the device 50 and the tube 52 relative to the patient.

An axel 80 extends through the center sections 76, 78 adjacent the first ends 56, 66 of each of the first and second arms 54, 64 that is structured to provide the pivotal association between the first and second arms 54, 64. Because the first and second arms 54, 64 are curvilinear in shape as seen in the lateral view of FIG. 6, placement of the axel 80 must allow the arms 54, 64 to move without interfering with each other and to achieve the widest amount of opening between them when the device 50 is opened. Hence, the axel 80 is located adjacent the first ends 56, 66 of each of the first and second arms 54, 64. This construction is designed to allow the first and second arms 54, 64 to freely pivot about the axel 80 and to freely move relative to one another. This eliminates the need to use flexible or compliant material as used in the clamp 20 described above. Moreover, it eliminates the use of force by the user to overcome the biasing of the arms together when a user attempts to pry the arms open to insert the tube, and it eliminates the use of force by the user to overcome biasing of the arms apart as the user squeezes them together. With the design shown in FIGS. 4-6, the arms can be easily rotated relative to one another to permit insertion of the tube 52, adjustment of the tube 52, and clamping of the tube 52.

As shown in this representative implementation of the present disclosure, the first connecting component 60 is a post and the second connecting component 70 is a C-shaped opening that is sized and shaped to snap over the post 60 with frictional engagement and retain the device 50 in the closed configuration. This provides a secure lock-and-unlock mechanism for multiple use and adjustment.

Preferably, the first clamp component 62 comprises a U-shaped first portion of the opening 74 that opens towards a first direction, and the second clamp component 72 comprises a U-shaped second portion of the opening 74 that opens towards a second direction that is opposite to the first direction when the first and second arms 54, 64 are in the closed configuration. Ideally, one or preferably both of the U-shaped openings 62, 72 have a gripping structure, such as a taberculated interior surface 63, 73 or a rough textured surface 63, 73, to grip the endotracheal tube when the device 50 is in the closed position. The first and second arms 54, 64 will have a smooth rounded exterior outer surface to provide a non-abrasive interface with the skin and mouth of the patient to preserve skin integrity and further increase comfort.

The opening 74 with gripping surface, such as taberculations 63, 73 will be a predetermined size so as to specifically fit the external diameters of various endotracheal tube 52 sizes commonly used in the Neonatal Intensive Care Unit (NICU) with a tight fit, i.e., a fit that resists movement of the tube 52 relative to the opening 74 in the device 50. More specifically, the external diameters include without limitation 3.6 mm, 4.3 mm, 4.9 mm, and 5.6 mm, which are generally associated with internal diameters of 2.5 mm, 3.0 mm, 3.5 mm, and 4.0 mm respectively. Ideally, the device 50 will be clearly marked with appropriate numeric size indicating the tube 52 size that it fits along with an individual color scheme for easy visual recognition. Ideally, the device 50 will be constructed from clear-colored plastic so that when the tube 52 is placed, the markings or the depth can be easily visualized.

Because the tube size used on infant patients is based on weight and not age, theoretically one could have a very small pediatric patient that is using a 4.0 sized tube, which is the largest tube size commonly used in infants. The depth of the tube 52 describes how deep the tube 52 is placed into the trachea. One can see this on a radiograph and describe the location by describing the location where the tube 52 terminates by the corresponding thoracic vertebrae number (for example, the tube 52 is at T2 or T3). It can also be described by looking at the number on the tube 52 (the tube 52 has pre-marked lengths, starting at the end of the tube 52 marked as 0 cm, then up the tube up to 15 cm). When a patient is intubated, one can also look at the number at the mouth line (gum line or lip line) and use that as a marker of where the tube 52 should be secured.

As can be seen in FIGS. 4-6, the first ends 56, 66 and second ends 58, 68 of each of the first and second arms 54, 64 are enlarged with respect to the respective center sections 76, 78 of the first and second arms 54, 64 to provide anchor points for the tape. Because the area around the mouth can be moist, which can inhibit adhering of the adhesive on the tape to the first and second arms 54, 64, the enlarged first ends 56, 66 and second ends 58, 68 cooperate to provide not only a greater surface area but also to provide a necked down area of reduced diameter for the tape to grip the device 50.

As will be appreciated from the foregoing, the device 50 of the present disclosure can be utilized in a system that includes the endotracheal tube 52, the device 50, and a securing or fastening means, such as tape, for fastening the device 50 to the patient and to hold the endotracheal tube in place when the endotracheal tube is placed into the trachea of the patient.

At this time, the tube 52 is secured using the device 50 (known as a Secure Endotracheal Tube Holder (SETH)), with the device 50 positioned horizontally across the mouth, flush against the lips, and taped to the mouth and cheeks as to maintain proper positioning.

Once the tube 52 is secured using the SETH device 50, the neonate will be transported from the delivery room straight to the Neonatal Intensive Care Unit (NICU) for further management. During transport, the end of the tube 52 is connected to an oxygen source and the respiratory therapist administers a breath every few seconds. Once in the NICU, a radiograph of the chest will be shot to confirm proper placement of the tube 52. Once satisfactory placement is confirmed, it may require adjustment in depth, i.e., pull back or push in the tube 52 as needed. This can be easily done by undoing the tape, opening the device 50 clasp or connecting components 60, 70, adjusting the tube 52, then reconnecting the device 50 and re-taping it in the corrected position based on the chest radiograph. At this time, the end of the tube 52 will connected to a ventilator with the appropriate setting based on the neonate's respiratory requirements.

Each SETH device 50 will come individually prepackaged in a plastic wrap, and they will come in four different sizes to fit the most commonly used tube sizes in neonates (2.5/3.0/3.5/4.0 mm). Together with the tubes, the SETH device 50 will be stocked in most frequently used areas such as the delivery room, operating room and in the code carts in the NICU.

### Method of Use

First, the infant patient is intubated by placing the endotracheal tube 52 in the trachea. The SETH device 50 is removed from package and clipped onto the tube 52 so that the tube 52 goes through the opening 74. The device 50 should be positioned horizontally across the mouth as if baby were biting down on a bar.

A skilled medical practitioner in this field should be able to visualize depth of the tube 52 (numerical number on the side of the tube 52) with the SETH device 50 clipped into place.

Cloth tape will be used to secure the device 50 along with the tube 52 onto the patient's cheeks. Two pieces of 1-inch cloth tape will be used. Each piece will be cut approximately 3 inches long. Then each piece will be cut in half longitudinally, leaving a ½ inch unsplit or uncut piece at the end. This will form an approximate V shape. There will be a piece of “Replicare thin™” or similar material to provide a barrier against the tape to the skin on each side of the cheeks. Then the tape will be attached to this barrier with the spilt side towards the middle of the mouth, towards the tube 52 and the device 50. The top strip will encircle the shaft of the tube 52 while the bottom strip will be secured around the opposite end of the tube 52. This will be repeated on the other side.

Placement of the tube 52 will be confirmed by radiograph. The position and depth of the tube 52 can be adjusted based on radiographic imaging and then by unclipping the SETH device 50 and re-taping into the new position as described above.

The various implementations described above can be combined to provide further implementations. Aspects of the implementations can be modified, if necessary to employ concepts of the various patents, applications and publications to provide yet further implementations.

These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the claims are not limited by the disclosure.

1. A device for use with an endotracheal tube, comprising:
a first arm having a curvilinear planform shape, a first end, an opposing second end having a first connecting component, and a first clamp component located between the first end and the second end; and
a second arm having a curvilinear shape that is sized and shaped to enable the second arm to be parallel to the first arm in a nested relationship, a first end, and an opposing second end having a second connecting component that is capable of cooperating with the first connecting component to releasably connect the second end of the second arm to the second end of the first arm, the second arm further including a second clamp component located between the first end and the second end that is capable of aligning with the first clamp component to form an opening sized and shaped to clamp the endotracheal tube in place in the opening, the second arm rotatably coupled to the first arm to enable relative movement of the first and second arms between an open position in which the second arm is not in a nested relationship with the first arm and a closed position in which the first and second arms are in the nested relationship and in which the first connecting component is connected to the second connecting component to hold the device in the closed position.

2. The device of claim 1 wherein at least one of the first and second clamp components has a taberculated gripping surface to grip the endotracheal tube when the device is in the closed position.

3. The device of claim 1 wherein the first arm is pivotally associated with the second arm at a connection point adjacent to the first ends of the first and second arms to enable the second end of the first arm to freely pivot and move into and out of connecting engagement with the second end of the second arm.

4. The device of claim 3 wherein the first connecting component comprises a post and the second connecting component comprises a C-shaped opening that is sized and shaped to snap over the post with frictional engagement and retain the device in the closed position.

5. The device of claim 1 wherein the first and second arms each have a substantially linear center section that connects the respective first ends to the second ends, and the device further comprises an axel extending through the center section adjacent the first end of each of the first and second arms that provides a pivotal association between the first and second arms.

6. The device of claim 1 wherein the first clamp component comprises a first portion of the opening that opens towards a first direction and the second clamp component comprises a second portion of the opening that opens towards a second direction that is opposite the first direction when the first and second arms are in the closed position.

7. The device of claim 5 wherein the first and second ends of each of the first and second arms are enlarged with respect to the respective center sections of the first and second arms to provide anchor points.

8. A system for intubating a patient, the system comprising:
an endotracheal tube;
a device for attachment to the endotracheal tube that includes:
a first arm having a curvilinear planform shape, a first end, an opposing second end having a first connecting component, and a first clamp component located between the first end and the second end;
a second arm having a curvilinear shape that is sized and shaped to enable the second arm to be parallel to the first arm in a nested relationship, a first end, and an opposing second end having a second connecting component that is capable of cooperating with the first connecting component to releasably connect the second end of the second arm to the second end of the first arm, the second arm further including a second clamp component located between the first end and the second end that is capable of aligning with the first connecting component to form an opening sized and shaped to clamp the endotracheal tube in place in the opening, the second arm rotatably coupled to the first end of the first arm to enable movement of the first and second arms between an open position in which the second arm is not in a nested relationship with the first arm and a closed position in which the first and second arms are in the nested relationship and in which the first connecting component is connected to the second connecting component to hold the device in the closed position; and
means for holding the endotracheal tube and the device for attachment to the endotracheal tube to the patient with the endotracheal tube placed into a trachea of the patient.

9. The system of claim 8 wherein at least one of the first and second clamp components have a gripping structure to grip the endotracheal tube when the device is in the closed position.

10. The system of claim 8 wherein the first arm is pivotally associated with the second arm at a connection point adjacent to the first ends of the first and second arms to enable the second end of the first arm to move into and out of connecting engagement with the second end of the second arm.

11. The system of claim 8 wherein the first connecting component comprises a post and the second connecting component comprises a C-shaped opening that is sized and shaped to snap over the post and retain the device in the closed position.

12. The system of claim 10 wherein the first and second arms each have a substantially linear center section that connects the respective first ends to the second ends, and the device further comprises an axel extending through the center section adjacent the first end of each of the first and second arms that provides the pivotal association between the first and second arms.

13. The system of claim 8 wherein the first clamp component comprises a first portion of the opening that opens towards a first direction and the second clamp component comprises a second portion of the opening that opens towards a second direction that is opposite to the first direction when the first and second arms are in the closed position.

14. The system of claim 12 wherein the first and second ends of each of the first and second arms are enlarged with respect to the respective center sections of the first and second arms to provide anchor points.
